(19) United States
(12) Patent Application Publication
(10) Pub. No.: US 2009/0062855 A1
(43) Pub. Date: Mar. 5, 2009

(54) BABY NEBULIZER

(76) Inventors: Tammy L. Lemery, Apache Junction, AZ (US); Anthony J. Crane, Apache Junction, AZ (US)

Correspondence Address: NW-IP-LAW, 6039 E. Grant RD., Tucson, AZ, 85712, US

(21) Appl. No.: 11/846,675

(22) Filed: Aug. 29, 2007

(51) Int. Cl. A61J 17/00; A61M 11/00

(52) U.S. Cl. 606/236; 128/200.14; 606/234

(57) ABSTRACT

The present invention features a pacifier comprising (a) a nipple with a first end and a second end, the first end has a perforation; (b) an attachment device having a first end and a second end, the first end of the attachment device fluidly mates with the second end of the nipple, the second end of the attachment device has a port to receive a tube, the attachment device further has a divider member disposed therein; and (c) a seal system having a seal member and a spring member.

### FIELD OF THE INVENTION

The present invention is directed to a baby nebulizer that includes a pacifier and a tube to enable a person to administer medicine to an infant or child without causing the infant or child to resist administration of the medicine, while simultaneously using the pacifier as a standard pacifier.

### BACKGROUND OF THE INVENTION

Persons administering medicine to infants or children often wish to administer the medicine in a non-confrontational way. They also may wish to have an easy way to administer the medicine, while being able to use the administration tool for another means, such as a standard pacifier. One solution is a medicine container that is attached to a face mask. A problem with this solution is that a child or infant can see the medicine being administered and may become agitated. Another problem with this solution is the medicine container and face mask cannot be used for any further uses.

Therefore, there is a need for an improved baby nebulizer, where a person may administer medicine to a child without the child knowing and resisting the administration, while simultaneously being able to use the baby nebulizer for a standard pacifier.

### SUMMARY OF THE INVENTION

The present invention features a pacifier comprising (a) a nipple with a first end and a second end, the first end has a perforation; (b) an attachment device having a first end and a second end, the first end of the attachment device fluidly mates with the second end of the nipple, the second end of the attachment device has a port to receive a tube, the attachment device further has a divider member disposed therein; and (c) a seal system having a seal member and a spring member. In some embodiments, the seal member comprises a foot of the seal at one end and a head of the seal at the other end and an opening through which an atomized medication passes from the tube into the nipple, wherein the spring member is compressibly positioned between the foot of the seal member and the divider member. In some embodiments, when there is no tube inserted into the port of the attachment device, the spring member expands and pushes onto the foot of the seal in a direction away from the divider, causing the seal member to glide through a hole of the divider member in a direction away from the nipple, whereby the head of a seal member mates with the divider member and thereby blocks the atomized medicine from entering into the nipple. In some embodiments, when the tube is inserted into the port of the attachment device, the tube pushes the foot of the seal member upwardly towards the nipple, compressing the spring member, whereby the opening of the seal member is exposed to the inside of the nipple to allow the atomized medicine to flow from the port through the opening and into the nipple.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1 is an illustration of a baby nebulizer as used according to the invention, including a pacifier, a tube, and a medicine vial.

FIG. 2 is an illustration of a baby nebulizer according to the invention, including a pacifier and a tube.

FIG. 3 is a side illustration of a cross-section of a pacifier in an open position according to the invention. The atomized medicine (cloud of dots) fill the nipple and exit the perforations. A first end of the nipple is on the right side of the drawing (the baby sucks on the first end). A second end of the nipple is on the left side, fluidly mating with the attachment device.

FIG. 4 is a side illustration of a cross-section of a pacifier in a closed position according to the invention.

### DESCRIPTION OF PREFERRED EMBODIMENTS

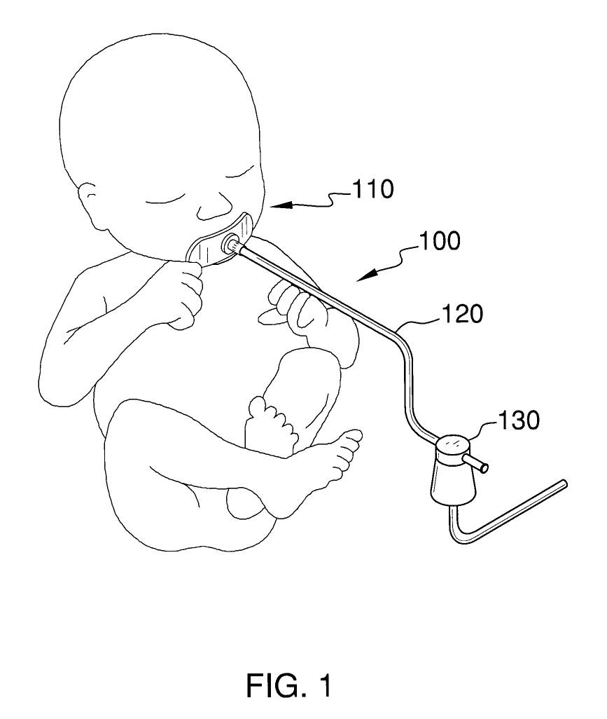

Referring now to FIG. 1, baby nebulizer 100 comprises pacifier 110, tube 120, and medicine vial 130. In the illustrated embodiment of FIG. 1, baby nebulizer 100 is shown as used on an infant. Generally, the medicine vial 130 comprises an “atomizer” (i.e., a “nebulizer”). An atomizer is a device used to administer a medication in the form of a liquid mist to the airways. It is commonly used in treating cystic fibrosis, asthma, and other respiratory diseases. An “atomizer” pumps air or oxygen through a liquid medicine to turn it into a vapor, which is then inhaled by the patient. In the present invention, the vapor is fed into a tube 120.

An atomizer is usually loaded with a medicine in the form of a concentrated liquid, sometimes viscous. In some embodiments, the medicines are steroids, e.g., bronchodilators such as Albuterol. One of the reasons that these medicines, e.g., steroids, are inhaled instead of ingested is to limit their effect to the lungs and respiratory system only, so as to prevent a saturation of steroid throughout the body which would be deleterious.

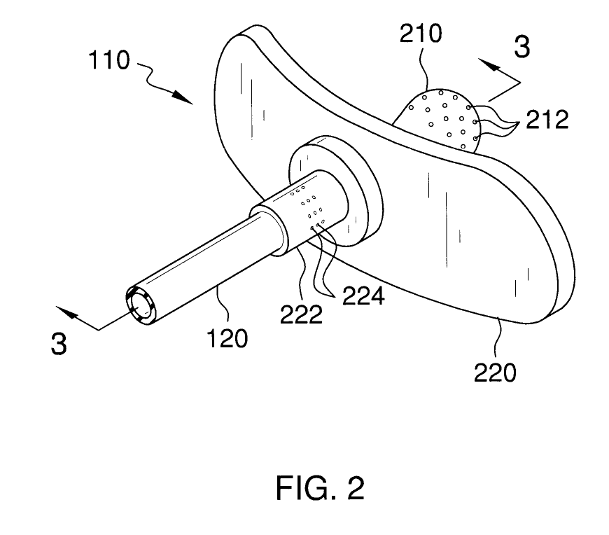

Referring now to FIG. 2, baby nebulizer 100 comprises pacifier 110 and tube 120. In the illustrated embodiment of FIG. 2, pacifier 110 comprises nipple 210 and mouthpiece 220. In the illustrated embodiment of FIG. 2, nipple 210 comprises first plurality of perforations 212. In some embodiments, the nipple 210 comprises a variety of flavors so the infant or child will not taste medicine being administered. The flavors can be coated onto the nipple by, for example, dipping the nipple 210 into a flavored syrup known to one of ordinary skill. In the illustrated embodiment of FIG. 2, mouthpiece 220 comprises attachment device 222 and, optionally, second plurality of perforations 224.

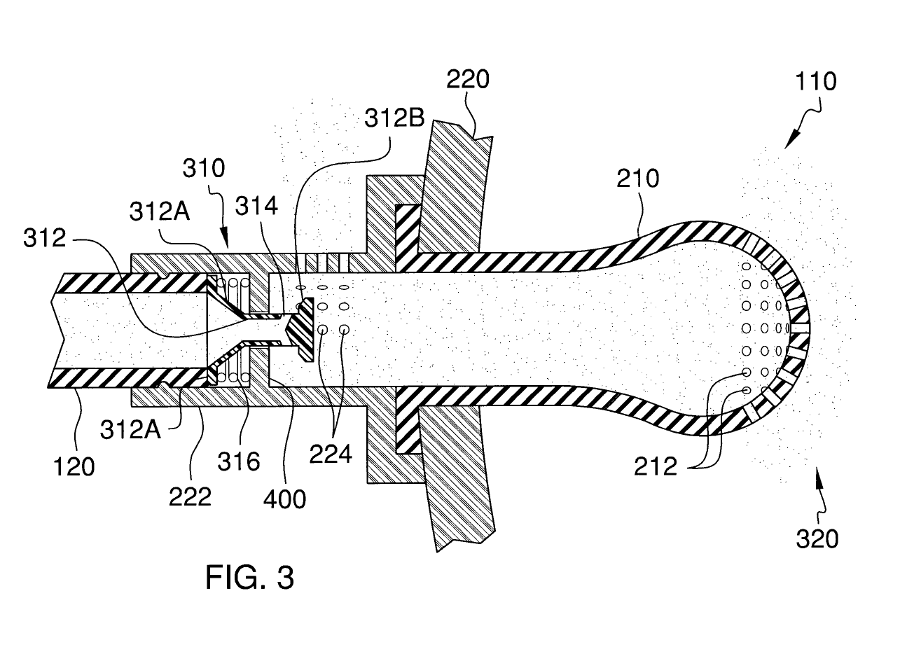

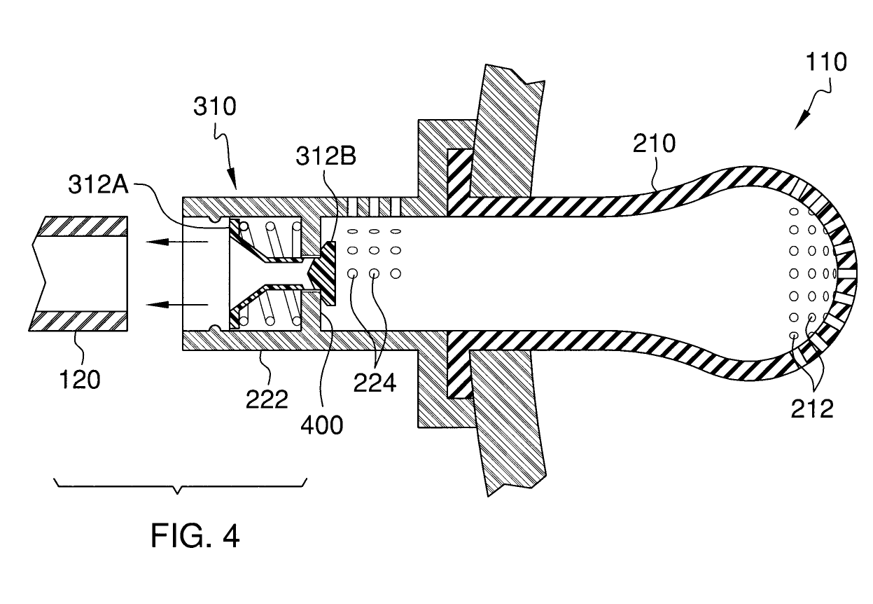

In the illustrated embodiment of FIG. 2, tube 120 is inserted into attachment device 222 to start the flow of medicine from medicine vial 130 (see FIG. 1). In the illustrated embodiment of FIG. 2, second plurality of perforations 224 allow for excess medicine to flow outwardly before reaching the mouth of the user. In some embodiments, the present baby nebulizer does not comprise a second plurality of perforations 224. In the illustrated embodiment of FIG. 2, tube 120 is releasably attached to attachment device 222. A user may pull tube 120 out of attachment device 222 to stop the flow of medicine. When a user pulls tube 120 out of attachment device 222, seal member 310 will move into a position where the opening 314 will be blocked, and allow pacifier 110 to be used as a regular pacifier known to one skilled in the art (see FIGS. 3 and 4).

In the illustrated embodiment of FIG. 2, tube 120 comprises a standard plastic tube of various diameters known to one skilled in the art. In the illustrated embodiment of FIG. 2, attachment device 222 comprises a tubular member having an inside diameter that is substantially equal to the outside diameter of tube 120. By “substantially equal,” Applicants mean a diameter that is equal +/−0.1 millimeters. In the illustrated embodiment of FIG. 2, mouthpiece 220 comprises a rigid material selected from the group consisting of plastic, an engineering plastic, or combinations thereof. In certain embodiments, mouthpiece 220 comprises an oval shape. In other embodiments, mouth piece 220 comprises a circular shape. In the illustrated embodiment of FIG. 2, nipple 210 comprises a flexible material, such as rubber.

Referring now to FIG. 3, baby nebulizer 100 comprises pacifier 110 and tube 120. In the illustrated embodiment of FIG. 3, pacifier 110 comprises nipple 210, a divider 400 with a hole where the seal member slides through, a spring member, and mouthpiece 220, wherein the seal member comprises a foot of the seal member 312a, a head of the seal member 312b, and an opening. In the illustrated embodiment of FIG. 3, nipple 210 comprises first plurality of perforations 212. In the illustrated embodiment of FIG. 3, mouthpiece 220 comprises attachment device 222 and, optionally, a second plurality of perforations 224. In the illustrated embodiment of FIG. 3, pacifier 110 comprises seal system 310. In the illustrated embodiment of FIG. 3, seal system 310 comprises seal member 312 and fitting 316, wherein the seal member 312 further comprises an opening 314.

When tube 120 is attached to attachment device 220, the tube 120 pushes against the seal member 312 in a first direction, wherein the spring member 316 is compressed (between the foot of the seal member 312a and the divider 400). When the seal member 312 is forced upwardly in the first direction, the opening 314 of the seal member 312 becomes exposed to the internal of the nipple 210, wherein the internal of the tube 120 is now in fluid communication with the internal of the nipple 210. In other words, when seal member 312 is forced upwardly, opening 314 is in an open position to allow atomized medicine 320 to flow outwardly into nipple 210.

When a user wishes to stop the flow of medicine 320, the user can pull tube 120 out of attachment device 222, whereby the spring member expands and pushes on the foot of the seal member 312a in a second and opposite direction. As the seal member 312 moves out of nipple 210, opening 314 is in a closed position (e.g., the opening 314 is blocked off by the divider) and medicine 320 is no longer able to flow through nipple 210 (see FIG. 4).

Referring now to FIG. 4, baby nebulizer 100 comprises pacifier 110 and tube 120. In the illustrated embodiment of FIG. 4, pacifier 110 comprises nipple 210 and mouthpiece 220. In the illustrated embodiment of FIG. 4, nipple 210 comprises first plurality of perforations 212. In the illustrated embodiment of FIG. 4, mouthpiece 220 comprises attachment device 222 and second plurality of perforations 224. In the illustrated embodiment of FIG. 4, pacifier 110 comprises seal system 310. In the illustrated embodiment of FIG. 4, seal system 310 comprises seal member 312 and spring member 316, wherein the seal member comprises an opening 314.

When tube 120 is attached to attachment device 220, spring member 316 is forced upward in a first direction, which forces seal member 312 upward into nipple 210. As seal member 312 is forced upward, opening 314 is in an open position to allow medicine 320 to flow outward into nipple 210.

When a user wishes to stop the flow of medicine 320, the user will pull tube 120 out of attachment device 222, and spring member 316 is moved in a second and opposite direction. As seal member 312 moves out of nipple 210, opening 314 is in a closed position and medicine 320 is no longer able to flow through nipple 210. In some embodiments, the spring member 316 comprises a compressible material (e.g., rubber, spring, etc.) that can be compressed when the tube 120 is pressed against the foot of the seal 312a, and the compressible material expands when the tube 120 no longer presses on the base of the seal 312a.

In some embodiments, the present invention features a pacifier 110 comprising (a) a nipple 210 with a first end and a second end, the first end has one or more perforations 212; (b) an attachment device 222 having a first end and a second end, the first end of the attachment device 222 fluidly mates with the second end of the nipple 210, the second end of the attachment device 222 has a port to receive a tube 120, the attachment device further has a divider member 400 disposed therein; and (c) a seal system 310 having a seal member 312 and a spring member 316. In some embodiments, the seal member comprises a fluid channel within, with one end of the channel exposed towards the tube 120 and the other end capable of being exposed toward the inside of the nipple 210 (i.e., the opening 314). In some embodiments, the opening 314 is located adjacent to the head of the seal 312b.

As shown on FIG. 3, the first end of the nipple 210 is on the right side of the drawing, and the second end of the nipple 210 is on the left, mating with the first end of the attachment device 222. The second end of the attachment device 222 has a port to receive the tube 120.

In some embodiments, the seal member 312 comprises a foot of the seal 312a at one end and a head of the seal 312b at the other end and an opening 314 through which an atomized medication passes from the tube 120 into the nipple. In some embodiments, the spring member 316 is compressibly positioned between the foot of the seal member 312a and the divider member 400. In some embodiments, when there is no tube 120 inserted into the port of the attachment device 222, the spring member 316 expands and pushes onto the foot of the seal 312a in a direction away from the divider 400, causing the seal member 312 to glide through a hole of the divider member 400 in a direction away from the nipple 210, whereby the head of a seal member 312b mates with the divider member 400 and thereby blocks the atomized medicine from entering into the nipple 210. In some embodiments, when the tube 120 is inserted into the port of the attachment device 222, the tube pushes the foot of the seal member 312 upwardly toward the nipple 210, compressing the spring member 316, whereby the opening of the seal member 314 is exposed to the inside of the nipple 210 to allow the atomized medicine to flow from the port through the opening 314 and into the nipple 210.

In some embodiments, the present invention features a pacifier 110 comprising a nipple 210 with a first end and a second end; the first end has one or more perforations 212, and a second end has a means for fluidly receiving a tube 120 which passes atomized medicine into the nipple 210.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A pacifier comprising:
(a) a nipple with a first end and a second end, the first end has a perforation;
(b) an attachment device having a first end and a second end, the first end of the attachment device fluidly mates with the second end of the nipple, the second end of the attachment device has a port to receive a tube,
the attachment device further has a divider member disposed therein; and
(c) a seal system having a seal member and a spring member, wherein the seal member comprises a foot of the seal at one end and a head of the seal at the other end and an opening through which an atomized medication passes from the tube into the nipple, wherein the spring member is compressibly positioned between the foot of the seal member and the divider member,
when there is no tube inserted into the port of the attachment device, the spring member expands and pushes onto the foot of the seal in a direction away from the divider, causing the seal member to glide through a hole of the divider member in a direction away from the nipple, whereby the head of a seal member mates with the divider member, thereby blocking the atomized medicine from entering into the nipple,
when the tube is inserted into the port of the attachment device, the tube pushes the foot of the seal member upwardly towards the nipple, compressing the spring member, whereby the opening of the seal member is exposed to the inside of the nipple to allow the atomized medicine to flow from the port through the opening and into the nipple.

2. The pacifier of claim 1 wherein the tube snugly fit into the port of the attachment device.

3. The pacifier of claim 2 wherein an opposite end of the tube is plugged into an atomizer.
